(19) United States
(12) Patent Application Publication
(10) Pub. No.: US 2021/0033615 A1
(43) Pub. Date: Feb. 4, 2021

(54) SCREENING METHOD FOR THE IDENTIFICATION OF CANCER THERAPEUTICS

(71) Applicant: Deutsches Krebsforschungszentrum Stiftung does Offentlichen Rechts, Heidelberg (DE)

(72) Inventors: Violaine Rosenstiel-Goidts, Graben-Neudorf (DE); Frederic Bethke, Aglasterhausen (DE); Jorg Balss, Dossenheim (DE); Emma Phillips, Heidelberg (DE)

(21) Appl. No.: 16/965,762

(22) PCT Filed: Jan. 25, 2019
(86) PCT No.: PCT/EP2019/051889

§ 371 Date: Jul. 29, 2020

(30) Foreign Application Priority Data
Jan. 29, 2018 (EP) 18153933.9

(51) Int. Cl. G01N 33/574; G01N 33/68

(52) U.S. Cl. CPC G01N 33/57484; G01N 33/6845; G01N 2500/02; G01N 2333/916; G01N 2333/91215

(57) ABSTRACT

The present invention pertains to a method for identifying anti-cancer compounds. The invention is based on the finding that a direct protein-protein interaction between 6-phosphofructo-2-kinase/fructose-2,6-biphosphatase 4 (PFKFB4) and (F-box protein 28) FBXO28 silences a ubiquitin E3 ligase activity of FBXO28 towards HIF1a. Interfering with this protein-protein interaction leads to a strong induction of HIF1a proteasomal degradation and cell death in tumors, and therefore, compounds screened according to the present invention harbour therapeutic potential for the treatment of proliferative diseases such as cancer. The invention provides a screening method for cancer therapeutics based on the interaction of PFKFB4 and FBXO28, as well medical applications thereof.

### FIELD OF THE INVENTION

The present invention pertains to a method for identifying anti-cancer compounds. The invention is based on the finding that a direct protein-protein interaction between 6-phosphofructo-2-kinase/fructose-2,6-biphosphatase 4 (PFKFB4) and (F-box protein 28) FBXO28 silences a ubiquitin E3 ligase activity of FBXO28 towards HIF1a. Interfering with this protein-protein interaction leads to a strong induction of HIF1a proteasomal degradation and cell death in tumors, and therefore, compounds screened according to the present invention harbour therapeutic potential for the treatment of proliferative diseases such as cancer. The invention provides a screening method for cancer therapeutics based on the interaction of PFKFB4 and FBXO28, as well medical applications thereof.

### DESCRIPTION

Cancer is a major lethal disease for humans and is caused by physiologically-uncontrolled cell proliferation which affects normal physiological conditions of human body resulting in serious pathological reactions often leading to death. Although tremendous efforts on cancer studies and treatments have been made, presently, cancer is still the major cause of death to humans. There are multiple approaches to treat cancer patients including surgery, radiation therapy and chemotherapy.

Neoplastic cells preferentially utilize glycolysis to satisfy their increased needs for energy and biosynthetic precursors. The PFKFB enzymes (PFKFB1-4) synthesize fructose-2,6-bisphosphate (F2,6BP). F2,6BP activates 6-phosphofructo-1-kinase (PFK-1), an essential control point in the glycolytic pathway. Until recently, the PFKFB3 isozyme has been considered the principal source of the increased F2,6BP observed in cancer cells. However, new evidence indicates the co-expression of several PFKFB isozymes in transformed and untransformed tissues, as well as increased expression of the PFKFB4 isoform in several neoplastic cell lines and in tumors.

There is a continuing need in the art to identify agents with therapeutic potential for the treatment of proliferative disorders. The identification of compounds is urgently needed in order to allow the further development of therapies from the laboratory into the clinical practise. The more agents are known in the art to potentially be useful in therapy, the higher is the probability of novel successful treatment options in the future.

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

This problem is solved in a first aspect by a method for the identification of a compound which is useful as a medicament for the treatment of cancer, the method comprising the steps of:
(a) Providing a candidate compound,
(b) Providing a protein complex comprising PFKFB4 and FBXO28, or fragments or derivatives thereof, wherein PFKFB4 and FBXO28, or the fragments or derivatives thereof, are in direct protein-to-protein interaction with each other (such as binding each other),
(c) Contacting said candidate compound with the protein complex comprising PFKFB4 and FBXO28, or fragments or derivatives thereof, and
(d) Determining whether contacting in (c) results in a change of protein-protein interaction between PFKFB4 and FBXO28, or the fragments or derivatives thereof, optionally by comparison to a control,

wherein in the event of a reduction of protein-protein interaction between PFKFB4 and FBXO28, or the fragments or derivatives thereof, as determined in step (d), the candidate compound is useful as a medicament for the treatment of cancer.

In the context of the present invention the term “PFKFB4” refers to 6-phosphofructo-2-kinase/fructose-2,6-biphosphatase 4 enzyme. The term shall encompass the human version of this protein, but also its homologs, in particular in mouse or rat. Information on PFKFB4 can be derived from the human gene names webpage (https://www.genenames.org/) which the accession number HGNC:88₇₅. The PFKFB4 protein is accessible on the UniProt webpage under the accession number Q16877, and is further, at least the human version, shown in SEQ ID NO: 1 (isoform 1). All other isoforms of the protein shall also be encompassed by the present invention.

In the context of the present invention the term “FBXO28” refers to F-box protein 28. The term shall encompass the human version of this protein, but also its homologs, in particular in mouse or rat. Information on FBXO28 can be derived from the human gene names webpage (https://www.genenames.org/) which the accession number HGNC:29046. The FBXO28 protein is accessible on the UniProt webpage under the accession number Q9NVF7, and is further, at least the human version, shown in SEQ ID NO: 2 (isoform 1). All other isoforms of the protein shall also be encompassed by the present invention.

Hence, in some embodiments the derivative or fragment of PFKFB4 is characterized by its ability to be in protein-protein interaction with a full length FBXO28 protein.

Hence, in some embodiments the derivative or fragment of FBXO28 is characterized by its ability to be in protein-protein interaction with a full length PFKFB4 protein.

In this context, the ability to be in protein-protein interaction is preferably the ability of such a protein derivative or fragment to form an interaction that mimics the native protein-protein interaction between PFKFB4 and FBXO28. Such a native interaction is described herein in the example section. In some preferred embodiments the protein-protein interaction of PFKFB4 and FBXO28 is mediated and/or located in the phosphatase domain of PFKFB4, and/or a domain that is in spatial proximity to the phosphatase domain, wherein spatial proximity is within not more than ₅0 (preferably 40, 30, 20 or 10) amino acid residues N or C terminally apart from at least one amino acid residues located within the PFKFB4 phosphatase domain. Hence, preferably, any derivative or fragment of PFKFB4 preferably in embodiments retains the phosphatase domain mediating the interaction, and/or a domain that is in spatial proximity to the phosphatase domain, wherein spatial proximity is within not more than 50 (preferably 40, 30, 20 or 10) amino acid residues N or C terminally apart from at least one amino acid residues located within the PFKFB4 phosphatase domain, and wherein such domain is mediating the interaction. Most preferably in all aspects and embodiments, the derivative or fragment of PFKFB4 therefore is a PFKFB4 lacking the any domain or sequence but the phosphatase domain, or more exemplary lacking the kinase domain of PFKFB4.

In further preferred embodiments, the screening assay of the invention is a NanoBiT® Assay as described in detail in Dixon et al., (2016) “NanoLuc Complementation Reporter Optimized for Accurate Measurement of Protein Interactions in Cells” (ACS Chem. Biol., 11, 400-408; which is incorporated herein by reference in its entirety). However, also other assays known to the skilled artisan and suitable for determining protein-protein interactions may be subject of the present invention.

All references to the databases in context of the invention refer to their respective versions of Jan. 26, 2018.

In context of the invention also any variants or fragments of PFKFB4 and FBXO28 shall be encompassed if they have an amino acid with a sequence identity of at least 50, 60, 70, 80, 85, 86, 87, 88, 89, 90,91, 92, 93, 94, 95, 96, 97, 98, 99 or 100% compared to the amino acid sequence human PFKFB4 and FBXO28, respectively. Therefore to SEQ ID NO: 1 or respectively 2 as disclosed herein. Fragments or PFKFB4 and FBXO28 or there variants, shall preferably have a length of at least 30, 40, 50, 80, 100, 150, 200, 300 or more amino acids.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site). In particular for amino acid identity, those using BLASTP 2.2.28+ with the following parameters: Matrix: BLOSUM62; Gap Penalties: Existence: 11, Extension: 1; Neighboring words threshold: 11; Window for multiple hits: 40.

Fragments or derivatives or variants of PFKFB4 and/or FBXO28 can be easily tested for the ability to have a protein-protein interaction. Hence, the present invention also disclosed a method for determining whether a protein derivative, variant and/or fragment of PFKFB4 maintains the ability to interact (bind) directly to FBXO28 (preferably in full length version). The method comprises a step of contacting the candidate derivative, variant and/or fragment of PFKFB4 with (for example full length) FBXO28 protein, and detecting their interaction, by for example co-immuno precipitation. Also comprised is a method for determining whether a protein derivative, variant and/or fragment of FBXO28 maintains the ability to interact (bind) directly to PFKFB4 (preferably in full length version). The method comprises a step of contacting the candidate derivative, variant and/or fragment of FBXO28 with (for example full length) PFKFB4 protein, and detecting their interaction, by for example co-immuno precipitation.

The above method for testing protein variants/derivatives and/or fragments may in some embodiments form part of the afore described method for the identification of a compound which is useful as a medicament for the treatment of cancer, preferably if the method encompasses the use of any protein derivative, variant and/or fragment of PFKFB4 and/or FBXO28.

The method of the invention may in some embodiments comprising providing the (protein) complex within a biological assay cell, or is provided in a cell-free system. If the screening is performed in contact of a biological assay cell, then human cells such as HEK cells are preferably used.

In general there are no limits to then nature of the candidate compound usable in the screening of the invention. In preferred embodiments the candidate compound is selected from a small molecular compound (“small molecule”), a polypeptide, peptide, glyco-protein, a peptidomimetic, an antigen binding construct (for example, an antibody, an-tibody-like molecule or other antigen binding derivative, or an antigen binding fragment thereof), a nucleic acid such as a DNA or RNA, for example an antisense or in-hibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA.

The term “small molecule”, as used herein, refers to organic or inorganic molecules either synthesized or found in nature, generally having a molecular weight equal to or less than 1000 D, however the definition of small molecule is in some embodiments not limited by this number.

In the art various methods are known to assay protein-protein interactions (binding) qualitatively or quantitatively. The present invention shall encompass all such prior art known methods applicable by the person of ordinary skill. However, in some embodiments, the following approaches may be preferred:

(i) co-immuno precipitation of the interacting proteins,

(ii) a Förster resonance energy transfer (FRET),

(iii) yeast two hybrid assay,

(iv) protein-protein covalent cross-linking,

(v) mass spectroscopy,

(vi) affinity chromatography,

(vii) affinity blotting,

(viii) two-hybrid reconstruction

(ix) reporter gene assays (NanoBiT™),

(x) detection of HIF1a ubiquitylation,

(xi) detection of HIF1a degradation,

(xii) immunofluorescent based assays,

(xiii) detection of assay cell viability.

As used herein, the term “Protein-protein interaction” or any grammatically variants of this expression, refers to the close and stable association between proteins. It usually involves the formation of non-covalent chemical bonds such as hydrogen bonds. Direct interaction means that two proteins involved have close contact and form chemical bonds between them. Indirect interaction between two proteins occurs when they do not interact directly with each other but are bound together through interacting with other proteins which in turn interact directly or indirectly with each other. A preferred protein-protein interaction of the invention is mediated and/or located in the PFKFB4 phosphatase domain, or a domain that is in spatial proximity to the phosphatase domain, wherein spatial proximity is within not more than 50 (preferably 40, 30, 20 or 10) amino acid residues N or C terminally apart from at least one amino acid residues located within the PFKFB4 phosphatase domain.

The screening method of the invention is preferably performed in a non-human animal system, ex-vivo, or in-vitro. With respect to ex-vivo uses, the method is preferably done is cell free systems or alternatively in cell culture. In context of the latter, mammalian cells are preferable and in particular human cell lines may be used such as Human Embryonic Kidney cells (HEK).

The idea of the invention in general is the use of the method in the production and identification of anti-cancer therapeutics. Cancer in context of the invention is therefore preferably a PFKFB4-expressing cancer, preferably a cancer associated with an elevated expression of PFKFB4.

Preferably the cancer is any tumor or cancer disease, preferably selected from a liquid or solid tumor, and preferably is lung cancer, bladder cancer, ovarian cancer, uterine cancer, endometrial cancer, breast cancer, liver cancer, pancreatic cancer, stomach cancer, cervical cancer, lymphoma, leukemia, acute myeloid leukemia, acute lymphocytic leukemia, salivary gland cancer, bone cancer, brain cancer, colon cancer, rectal cancer, colorectal cancer, kidney cancer, skin cancer, melanoma, squamous cell carcinoma, pleomorphic adenoma, hepatocellular carcinoma, and/or adenocarcinoma. Preferred cancers are glioblastoma, breast, prostate or lung cancer.

In another aspect the problem of the invention is also solved by a method for the production of pharmaceutical composition, the method comprising identifying a compound with a method for the identification of a compound which is useful as a medicament for the treatment of cancer as described herein above, and formulating the compound as a pharmaceutical composition together with a pharmaceutically acceptable carrier and/or excipient.

As used herein the language “pharmaceutically acceptable” carrier, stabilizer or excipient is intended to include any and all solvents, solubilizers, fillers, stabilizers, binders, absorbents, bases, buffering agents, lubricants, controlled release vehicles, diluents, emulsifying agents, humectants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The pharmaceutical composition of the invention is formulated to be compatible with its intended route of administration. Examples of routes of administration include parenteral, e.g., intrathecal, intra-arterial, intravenous, intradermal, subcutaneous, oral, transdermal (topical) and transmucosal administration.

Further provided is a use of a compound identified according to a method for the identification of a compound which is useful as a medicament for the treatment of cancer as described herein above, for the production of a medicament for use in the treatment of cancer.

Yet a further aspect of the invention then pertains to a method of treating a cancer in a subject, the method comprising the step of interrupting in cell associated with the cancer in the subject the protein-protein interaction between PFKFB4 and FBXO28.

The cancer is preferably a cancer as defined herein above.

A subject according to the invention is preferably a mammal, preferably a human patient suffering from cancer and in need of a treatment.

The treatment method of the invention preferably comprises the administration to the subject of a therapeutically effective amount of a compound which specifically reduces the protein-protein interaction between PFKFB4 and FBXO28 in a cell associated with the cancer. The compound is preferably a compound as identified according to the herein disclosed invention.

The present invention will now be further described in the following examples with reference to the accompanying figures and sequences, nevertheless, without being limited thereto. For the purposes of the present invention, all references as cited herein are incorporated by reference in their entireties. In the FIG. s:

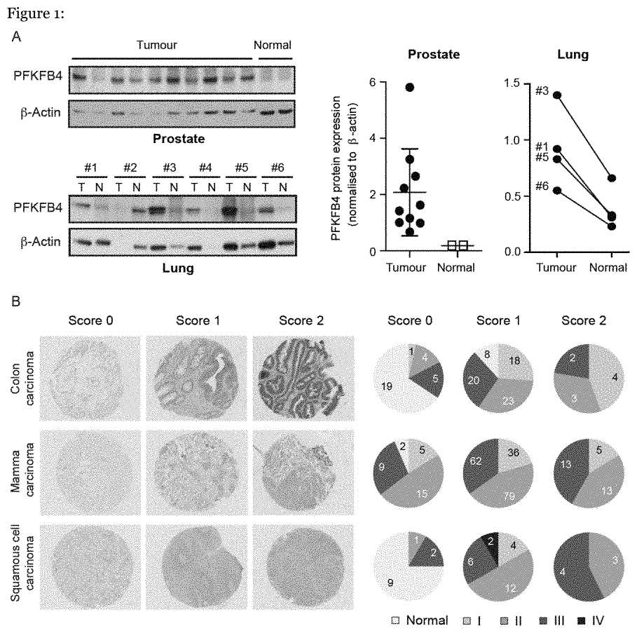

FIG. 1: TMA and Western blots

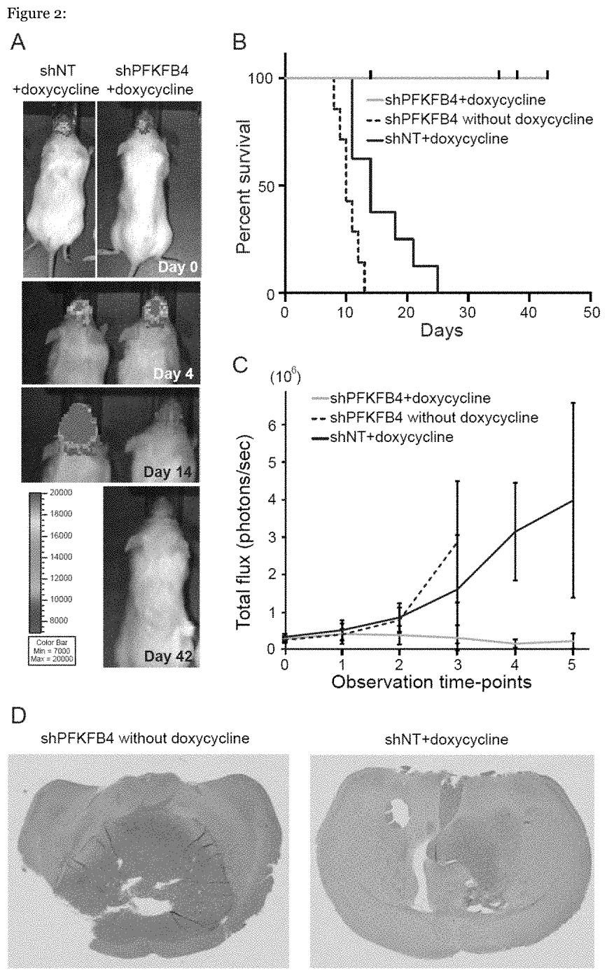

FIG. 2: (A) Luminescent image of animals stably ex-pressing the luciferase gene and treated with doxycycline for 6 weeks. (B) Kaplan Meier curves of the animals treated with doxycycline expressing shNT (black) or shPFKFB4 (grey) and non-treated animals (dashed). (C) Tumor size as determined by luminescent imaging, expressed in total flux (photons/second). (D) Scanned H and E stained sections of mouse brains from the non-treated and the shNT expressing groups using the Leica ImageScope. Whole brain, to-fold and 4o-fold magnification are presented.

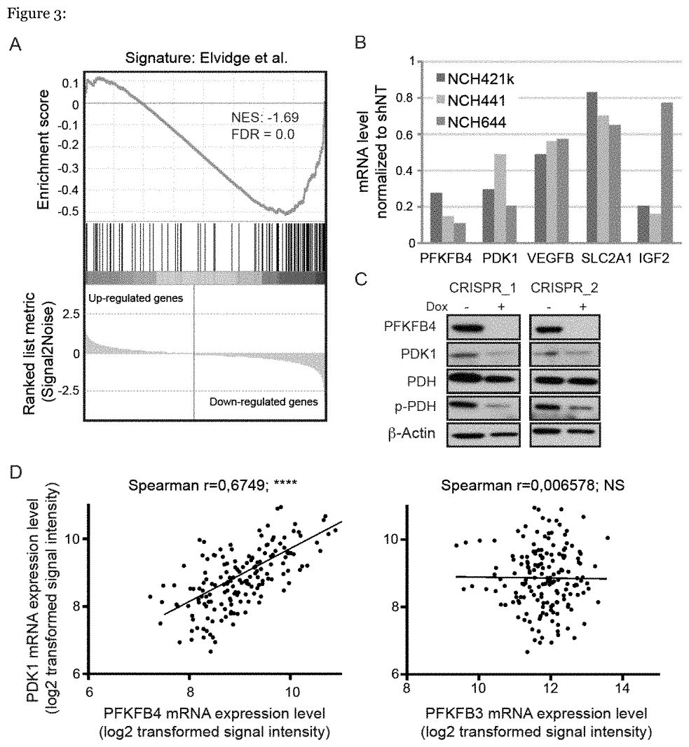

FIG. 3: PFKFB4 is involved in HIF transcriptional function

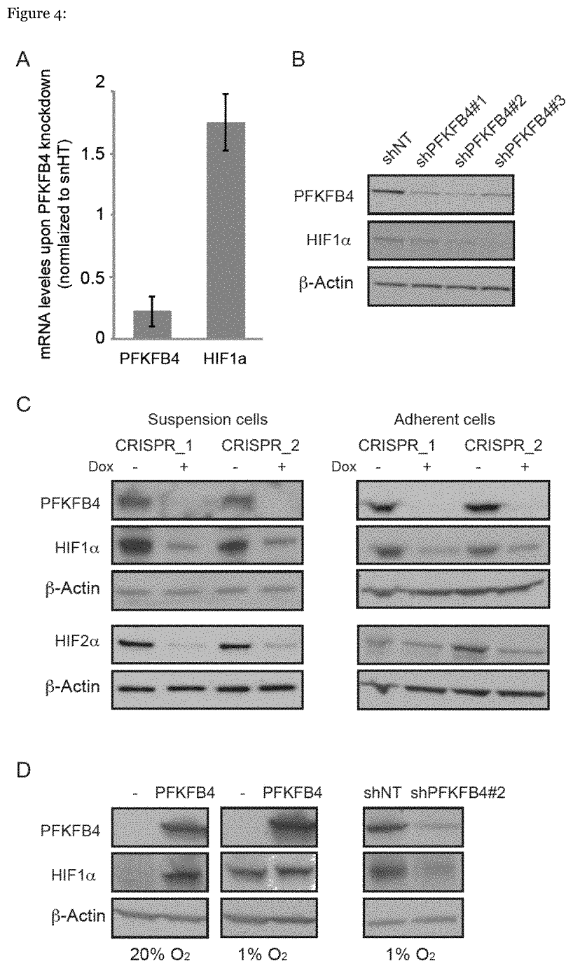

FIG. 4: Knockdown of PFKFB4 impairs HIF stability

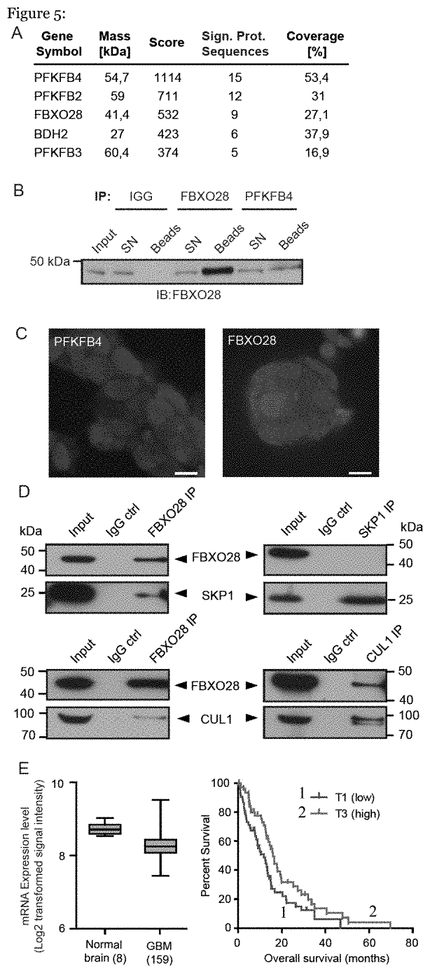

FIG. 5: FBXO28 interacts with PFKFB4

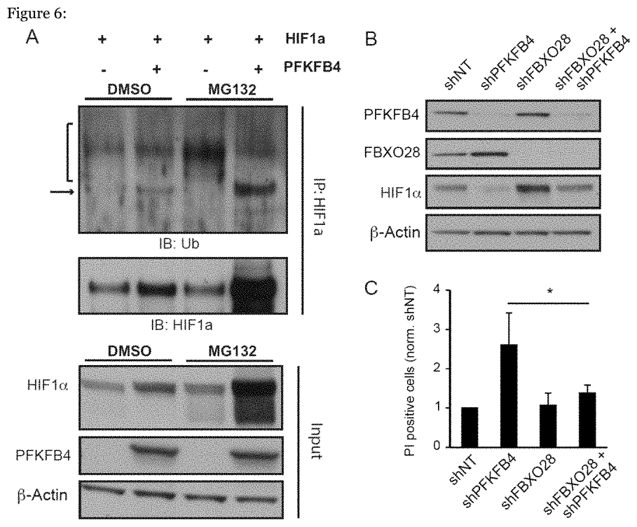

FIG. 6: FBXO28 is required for HIF1a stability

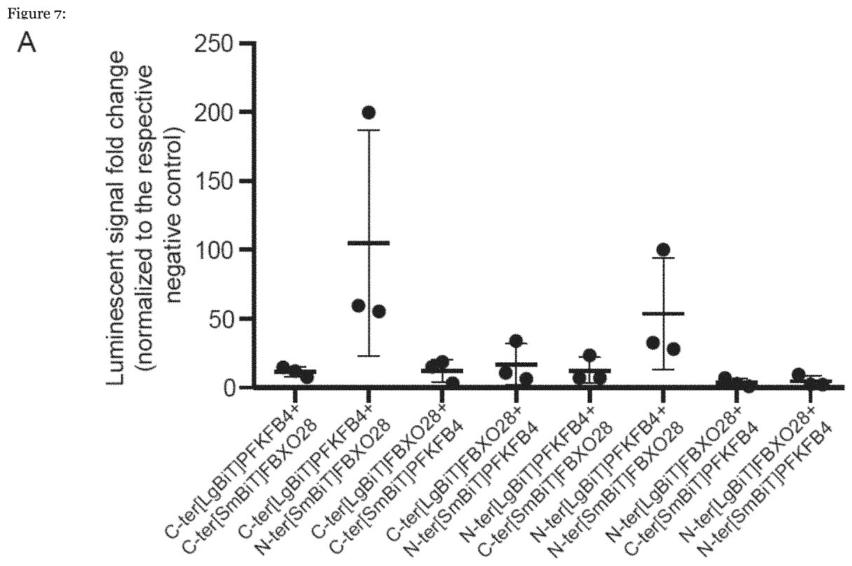

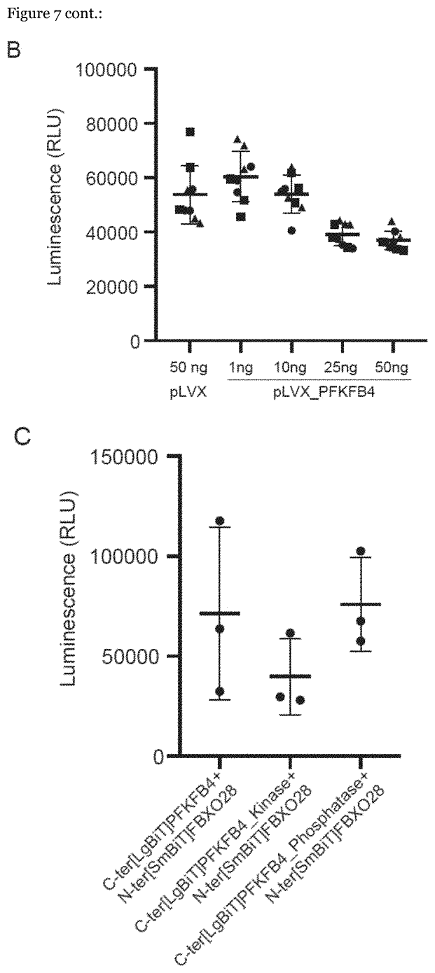

FIG. 7: shows a NanoBiT® assay for screening candidate compounds.

And in the sequences:

SEQ ID NO: 1
MASPRELTQNPLKKIWMPYSNGRPALHACQRGVCMTNCPTLIVMVGLPA
RGKTYISKKLTRYLNWIGVPTREFNVGQYRRDVVKTYKSFEFFLPDNEE
GLKIRKQCALAALRDVRRFLSEEGGHVAVFDATNTTRERRATIFNFGEQ
NGYKTFFVESICVDPEVIAANIVQVKLGSPDYVNRDSDEATEDFMRRIE
CYENSYESLDEDLDRDLSYIKIMDVGQSYVVNRVADHIQSRIVYYLMNI
HVTPRSIYLCRHGESELNLKGRIGGDPGLSPRGREFAKSLAQFISDQNI
KDLKVWTSQMKRTIQTAEALGVPYEQWKVLNEIDAGVCEEMTYEEIQDN
YPLEFALRDQDKYRYRYPKGESYEDLVQRLEPVIMELERQENVLVICHQ
AVMRCLLAYFLDKAAEQLPYLKCPLHTVLKLTPVAYGCKVESIFLNVAA
VNTHRDRPQNVDISRPPEEALVTVPAHQ
SEQ ID NO: 2
MAAAAEERMAEEGGGGQGDGGSSLASGSTQRQPPPPAPQHPQPGSQALP
APALAPDQLPQNNTLVALPIVAIENILSFMSYDEISQLRLVCKRMDLVC
QRMLNQGFLKVERYHNLCQKQVKAQLPRRESERRNHSLARHADILAAVE
TRLSLLNMTFMKYVDSNLCCFIPGKVIDEIYRVLRYVNSTRAPQRAHEV
LQELRDISSMAMEYFDEKIVPILKRKLPGSDVSGRLMGSPPVPGPSAAL
TTMQLFSKQNPSRQEVTKLQQQVKTNGAGVTVLRREISELRTKVQEQQK
QLQDQDQKLLEQTQIIGEQNARLAELERKLREVMESAVGNSSGSGQNEE
SPRKRKKATEAIDSLRKSKRLRNRK

### EXAMPLES

### Example 1

### Tumour-Specific Expression of PFKFB4

Recent studies have showed the importance of the key glycolysis gene PFKFB4 for the survival of different tumor cells in vitro and in vivo, highlighting its potential as therapeutic target. To investigate the endogenous protein expression level within different cancer entities, the inventors have developed a new PFKFB4 antibody that is suitable for Western blot and immunohistochemistry, allowing high-throughput staining of Tissue Microarrays (TMAs). As displayed in FIG. 1A, prostate tumor protein samples showed a significantly higher level of PFKFB4 expression than normal samples. Similarly, tumor and normal paired samples from lung cancer patients showed a marked difference of PFKFB4 expression level (FIG. 1A). As depicted in FIG. 1B, the level of PFKFB4 protein expression is ranging from not expressed to high expressed, normal tissues being mostly negative for PFKFB4. Interestingly, the level of expression correlated with the grading of the different tumor, irrespective of its tissue origin, suggesting that PFKFB4 could be involved in the maintenance and growth of tumors.

### Example 2

### Knockdown of PFKFB4 Impairs GSC Viability in Vivo

In order to verify the effect of PFKFB4 silencing on the tumor growth in vivo, we used a xenograft mouse model. For this purpose, we generated inducible expression constructs encoding an shRNA targeting PFKFB4 and a non-target shRNA as negative control. Stably transduced GSCs (NCH421k_TetONshPFKFB4 and NCH421k_TetONshNT) were treated for three days with doxycycline and the knockdown of PFKFB4 was characterized at the protein level. In order to allow the monitoring of the tumor growth in vivo, the inducible GSC lines were stably transduced with a luciferase-expressing construct.

Two groups of 8 and 14 animals were orthotopically transplanted with 100.000 NCH421k_Luc_TetONshNT and NCH421k_Luc_TetONshPFKFB4 cells respectively. The tumor growth expressed as luminescent signal was monitored twice a week. Apparition of the signal of sufficient size (ca. 200.000 flux/photon/second) determined the start of doxycycline treatment. Animals were randomly separated in three groups, namely shNT_dox treated (n=8), shPFKFB4_dox treated (n=7) and shPFKFB4_untreated (n=7). Pictures of the animals were taken twice a week (FIG. 2A). Animals expressing shPFKFB4 upon doxycycline treatment showed a significantly better survival than the control animals (non-treated animals and shNT expressing animals) (FIG. 2B).

Knockdown of PFKFB4 reduced significantly tumor size overtime as observed by bioluminescent intensity, ultimately leading to the loss of tumor cells about three weeks after treatment (FIG. 2C). Indeed, tumor could be observed in the brain of the sacrified sick animals (non-treated and doxycycline treated shNT) by H and E staining (FIG. 2D), while most of the shPFKFB4 doxycycline-treated mice were tumor-free. Residual tumors of shPFKFB4_dox treated animals still expressed PFKFB4, suggesting that the doxycycling induction of shPFKFB4 was not completely efficient in these tumors as confirmed by immunofluorescence.

### Example 3

### Impact of PFKFB4 Silencing on PDK1 Expression

As shown in vitro and in vivo, the impact of PFKFB4 silencing is particularly high for GSC viability. In order to determine its effect on the regulation of the expression of other genes, the gene expression of three different GSC lines (NCH421k, NCH441 and NCH644) transduced with pLKO_shPFKFB4 and pLKO_shNT was profiled. Interestingly, knockdown of PFKFB4 led to a decrease of the HIF1a gene signature, as determined by Gene Set Enrichment Analysis (GSEA) (FIG. 3A). Among the numerous known target of HIF1a the inventors identified, such as LDHA, CA9 and IGF2, the Pyruvate Dehydrogenase Kinase 1 (PDK1) showed the strongest reduction upon PFKFB4 knockdown, which was verified by qRT-PCR (FIG. 3B). PDKi is a key enzyme that regulates the fate of pyruvate within the glycolytic pathway by phosphorylating and thereby inhibiting the Pyruvate Dehydrogenase (PDH). In order to verify that the knockdown of PFKFB4 also affected the phosphorylation of PDKi's target, the inventors performed western blot analysis upon knockout of PFKFB4 using CRISPR. As depicted in FIG. 3C, the level of phosphorylated PDH decreased upon PFKFB4 knockdown.

Because the effect of PFKFB4 silencing on PDKi expression was significant, the endogenous expression of both genes was investigated in a cohort of glioblastoma patients (n=154), available from The Cancer Genome Atlas (TCGA). Notably, PDKi showed the highest expression correlation with PFKFB4 (R=0.67) (FIG. 3D), a phenomenon that seemed to be specific to that PFK2/FBP2 isoform. Indeed, none of the other isoforms that are expressed in glioblastoma samples showed any correlation with PDKi expression (FIG. 3D).

### Example 4

### PFKFB4 is Involved in the Regulation of HIF1a Protein Levels

As highlighted by the gene expression profiling of PFKFB4 knockdown GSC lines, PFKFB4 seems to be involved in the regulation of the expression of genes that are targets of the HIF1a transcription factor. In order to investigate the potential role of PFKFB4 on the regulation of HIF1a expression, qRT-PCR on PFKFB4 knockdown GSCs was performed. As shown in FIG. 4A, no decrease of HIF1a mRNA could be observed upon PFKFB4 knockdown. Indeed, it seems that upon silencing, HIF1a is upregulated at the mRNA level. However, the knockdown of PFKFB4 led to a strong decrease of HIF1a at the protein level (FIG. 4B), suggesting that PFKFB4 is involved post-transcriptionally in HIF1a regulation. This phenomenon was confirmed by knockout of PFKFB4 using two different CRISPR guide RNAs (FIG. 4C).

As GSCs are cultivated as neurospheres, which could lead to different level of oxygen and nutrient availability, thereby influencing the dependence on HIF1a transcription factor, the inventors performed a PFKFB4 knockout on adherent GSCs. Interestingly, even on these normoxic conditions, HIF1a is strongly expressed, suggesting that HIF1a protein levels are dependent on PFKFB4 expression, irrespective of the culture conditions (FIG. 4C). Furthermore, the overexpression of PFKFB4 in HEK293 cells led to the up-regulation of HIF1a, while its knockdown under hypoxic conditions decreased HIF1a protein levels (FIG. 4D).

### Example 5

### Identification of a New E3 Ubiquitin Ligase of HIF1a

As both proteins do not directly interact with each, the inventors performed mass spectrometry of immunoprecipitated PFKFB4 samples to find binding partners of PFKFB4 that could be involved in the mechanisms stabilizing HIF1a.

Proteins showing more than two signature sequences are listed in FIG. 5A. The binding of FBXO28 to PFKFB4 was verified by coIP and by Yeast-Two-Hybrid (FIG. 5B). In addition, the cytoplasmic localization of both proteins was verified by immunofluorescence (FIG. 5C). FBXO28 is a member of the F-Box protein family and is thought to be part of the SCF complex formed by SKPi, cullin and F-box proteins, as shown by coIP (FIG. 5D), acting as ubiquitin ligases. Interestingly, unlike PFKFB4, FBXO28 mRNA expression is decreased in glioblastoma as compared to normal brain and patients with a lower expression have a better survival (FIG. 5E).

### Example 6

### PFKFB4 Binds to FBXO28 to Inhibit HIF1a Ubiquitylation and Degradation

To verify the role of PFKFB4 in the ubiquitylation of HIF1a, the inventors performed immunoblotting using ubiquitin antibody on immunoprecipitated HIF1a upon MG132 treatment. As highlighted in FIG. 6A, the ubiquitylation of HIF1a is decreased in cells overexpressing PFKFB4. Next, the importance of the link between FBXO28 and for HIF1a protein expression and GSCs survival PFKFB4 was identified. In that respect, PFKFB4 was knocked down solely or in combination with FBXO28 silencing in GSCs and FACS analysis was performed. The effect on HIF1a protein level was verified by Western blot. As shown in FIG. 6B, knockdown of PFKFB4 decreased the protein level of HIF1a while silencing of FBXO28 alone did not reduce it. However, silencing of both FBXO28 and PFKFB4 in the GSCs rescued the level of HIF1a. The rescue was also seen at the phenotypic level (FIG. 6C).

Taken together, these results emphasize the potential role of PFKFB4 to protect HIF1a from the SCF complex, enabling the expression of HIF target genes in glioblastoma stem-like cells.

### Example 7

### Development of a Screening Method to Identify Small-Compound in-Hibitor to Inhibit PFKFB4 Function

The NanoBiT® assay from Promega is widely used to investigate protein-protein interactions. The NanoBiT Assay is described in detail in Dixon et al., (2016) “NanoLuc Complementation Reporter Optimized for Accurate Measurement of Protein Interactions in Cells” (ACS Chem. Biol., 11, 400-408; which is incorporated herein by reference in its entirety). The inventors adapted this method to develop a cellular screening assay in order to investigate the interaction of PFKFB4 with FBXO28. In that respect, both genes were cloned into pLVX vectors containing the four NanoBiT® versions (pLVX1.1-N[TK/LgBiT], pLVX2.1-N[TK/SmBiT], pLVX1.1-C[TK/LgBiT] and pLVX2.1-C[TK/SmBiT]), allowing the small and large BiT tags to be at the N- or C-terminal of both proteins of interest. As negative control, a vector containing a HaloTag protein cloned to the small BiT was used in combination with the large BiT cloned to either FBXO28 or PFKFB4.

All vectors were transfected in HEK293 cells and the combinations were tested as depicted in FIG. 7A. The luminescent signal was detected with the Nano-Glo® Luciferase Assay System from Promega. The interaction was considered positive if the signal was at least 10-fold of the respective negative control. The combination of the vector containing PFKFB4 tagged at the C-terminal with the Large BiT with the vector expressing FBXO28 tagged at the N-terminal with the small BiT (see FIG. 7A, 2^{nd} column) gave the highest signal and was therefore selected for further validation.

In order to verify the specificity of the assay, the inventors transfected HEK293 cells with the combination showing the best results (C-ter[LgBiT]PFKFB4+N-ter[SmBiT]FBXO28) together with increasing concentration of a vector overexpressing untagged PFKFB4. As shown in FIG. 7B, the signal given by the interaction of tagged PFKFB4 and FBXO28 decreased upon addition of untagged PFKFB4 which competes with tagged PFKFB4 to form the interacting complex. This is a clear indication that a candidate interacting compound (here represented by the untagged PFKFB4) in a screening assay would yield signals indicative for the impairment of the interaction between both proteins.

Finally, by transfecting different truncated versions of tagged PFKFB4 together with tagged FBXO28, the inventors were able to determine that the interaction is reduced if the phosphatase domain is removed and therefore, that the site of interaction between both proteins is most likely located within the phosphatase domain of PFKFB4 (FIG. 7C).

1. A method for the identification of a compound which is useful as a medicament for the treatment of cancer, the method comprising the steps of:
(a) Providing a candidate compound,
(b) Providing a protein complex comprising PFKFB4 and FBXO28, or fragments or derivatives thereof, wherein PFKFB4 and FBXO28, or the fragments or derivatives thereof, are in direct protein-to-protein interaction with each other (such as binding each other),
(c) Contacting said candidate compound with the protein complex comprising PFKFB4 and FBXO28, or fragments or derivatives thereof, and
(d) Determining whether contacting in (c) results in a change of protein-protein interaction between PFKFB4 and FBXO28, or the fragments or derivatives thereof, optionally by comparison to a control,
wherein in the event of a reduction of protein-protein interaction between PFKFB4 and FBXO28, or the fragments or derivatives thereof, as determined in step (d), the candidate compound is useful as a medicament for the treatment of cancer.

2. The method according to claim 1, wherein the complex is provided within a biological assay cell, or is provided in a cell-free system.

3. The method according to claim 1, wherein the candidate compound is selected from a small molecular compound (“small molecule”), a polypeptide, peptide, glycoprotein, a peptidomimetic, an antigen binding construct (for example, an antibody, antibody-like molecule or other antigen binding derivative, or an antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA.

4. The method according to claim 1, wherein the determining in step (d) involves at least one of:
(i) co-immuno precipitation of the interacting proteins,
(ii) a Förster resonance energy transfer (FRET),
(iii)yeast two hybrid assay,
(iv)protein-protein covalent cross-linking,
(v)mass spectroscopy,
(vi) affinity chromatography,
(vii) affinity blotting,
(viii)two-hybrid reconstruction
(ix)reporter gene assays (NanoBiT),
(x) detection of HIF 1 a ubiquitylation,
(xi) detection of HIF 1 a degradation,
(xii)immunofluorescent based assays,
(xiii)detection of assay cell viability.

5. The method according to claim 1, which is performed in a non-human animal system, ex-vivo, or in-vitro, preferably in a human cell line such as Human Embryonic Kidney cells (HEK).

6. The method according to claim 1, wherein the derivative or fragment of PFKFB4 is characterized by its ability to be in protein-protein interaction with a full length FBXO28 protein.

7. The method according to claim 1, wherein the derivative or fragment of FBXO28 is characterized by its ability to be in protein-protein interaction with a full length PFKFB4 protein.

8. The method according to claim 6, wherein the ability to be in protein-protein interaction is the ability of an interaction that mimics the native protein-protein interaction between PFKFB4 and FBXO28.

9. The method according to claim 1, wherein the cancer is a PFKFB4-expressing cancer, preferably a cancer associated with an elevated expression of PFKFB4, such as glioblastoma, breast, prostate or lung cancer.

10. A method for the production of pharmaceutical composition, the method comprising identifying a compound with a method according to claim 1, and formulating the compound as a pharmaceutical composition together with a pharmaceutically acceptable carrier and/or excipient.

11. Use of a compound identified according to the method of claim 1, for the production of a medicament for use in the treatment of cancer.

12. A method of treating a cancer in a subject, the method comprising the step of interrupting in cell associated with the cancer in the subject the protein-protein interaction between PFKFB4 and FBXO28.

13. The method according to claim 12, wherein the cancer is a cancer characterized by the expression of PFKFB4 and FBXO28.

14. The method according to claim 12, wherein the subject is a mammal, preferably a human patient suffering from cancer and in need of a treatment.

15. The method according to claim 12, wherein the method comprising the administration to the subject of a therapeutically effective amount of a compound which specifically reduces the protein-protein interaction between PFKFB4 and FBXO28 in a cell associated with the cancer.

16. A method of treating a cancer in a subject, the method comprising the step of interrupting in cell associated with the cancer in the subject the protein-protein interaction between PFKFB4 and FBXO28; wherein the compound is a compound as identified according to a method of claim 1.


SEQUENCE LISTING (2 sequences)

SEQ ID NO: 1 - protein, 469 aa, Homo sapiens
MASPRELTQNPLKKIWMPYSNGRPALHACQRGVCMTNCPTLIVMVGLPARGKTYISKKLTRYLNWIGVPTREFNVGQYRRDVVKTYKSFEFFLPDNEEGLKIRKQCALAALRDVRRFLSEEGGHVAVFDATNTTRERRATIFNFGEQNGYKTFFVESICVDPEVIAANIVQVKLGSPDYVNRDSDEATEDFMRRIECYENSYESLDEDLDRDLSYIKIMDVGQSYVVNRVADHIQSRIVYYLMNIHVTPRSIYLCRHGESELNLKGRIGGDPGLSPRGREFAKSLAQFISDQNIKDLKVWTSQMKRTIQTAEALGVPYEQWKVLNEIDAGVCEEMTYEEIQDNYPLEFALRDQDKYRYRYPKGESYEDLVQRLEPVIMELERQENVLVICHQAVMRCLLAYFLDKAAEQLPYLKCPLHTVLKLTPVAYGCKVESIFLNVAAVNTHRDRPQNVDISRPPEEALVTVPAHQ

SEQ ID NO: 2 - protein, 368 aa, Homo sapiens
MAAAAEERMAEEGGGGQGDGGSSLASGSTQRQPPPPAPQHPQPGSQALPAPALAPDQLPQNNTLVALPIVAIENILSFMSYDEISQLRLVCKRMDLVCQRMLNQGFLKVERYHNLCQKQVKAQLPRRESERRNHSLARHADILAAVETRLSLLNMTFMKYVDSNLCCFIPGKVIDEIYRVLRYVNSTRAPQRAHEVLQELRDISSMAMEYFDEKIVPILKRKLPGSDVSGRLMGSPPVPGPSAALTTMQLFSKQNPSRQEVTKLQQQVKTNGAGVTVLRREISELRTKVQEQQKQLQDQDQKLLEQTQIIGEQNARLAELERKLREVMESAVGNSSGSGQNEESPRKRKKATEAIDSLRKSKRLRNRK